(19) United States
(12) Patent Application Publication
(10) Pub. No.: US 2006/0247589 A1
(43) Pub. Date: Nov. 2, 2006

(54) Nature nappy/diaper and material constructions

(75) Inventor: Charishma M. W. Seneviratne, West Australia (AU)

Correspondence Address: NIXON & VANDERHYE, PC, 901 NORTH GLEBE ROAD, 11TH FLOOR, ARLINGTON, VA, 22203, US

(73) Assignee: SeNevens International Pty. Ltd., Belmont, West Australia (AU)

(21) Appl. No.: 10/547,005

(22) PCT Filed: Feb. 24, 2004
(86) PCT No.: PCT/AU04/00219

§ 371 Date: Aug. 25, 2005

(30) Foreign Application Priority Data
Feb. 25, 2003 (AU) 2003900879

(51) Int. Cl. A61F 13/15

(52) U.S. Cl. 604/368

(57) ABSTRACT

A multi-layered composition including an outer cover of natural fabric and cotton felt, and an absorbent core having a top layer of a natural feather fabric and a lower layer of a waterproof coating of wax. The absorbent core may further include as a second layer natural fluffed pulp and gel. The composition is adapted for use in a product such as a nappy or diaper, a personal product, clothing item or protective item incorporating the composition.

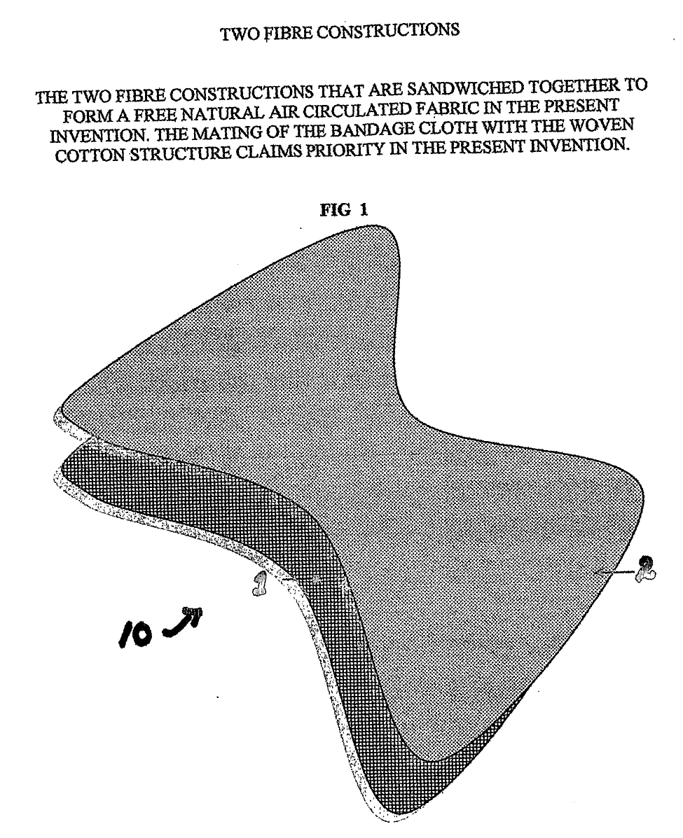

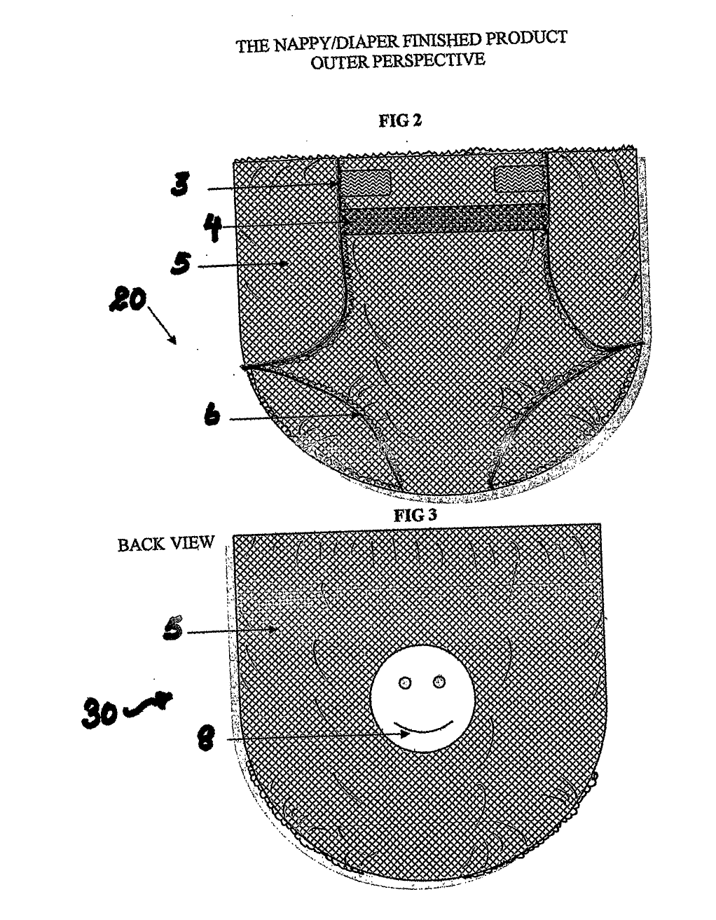

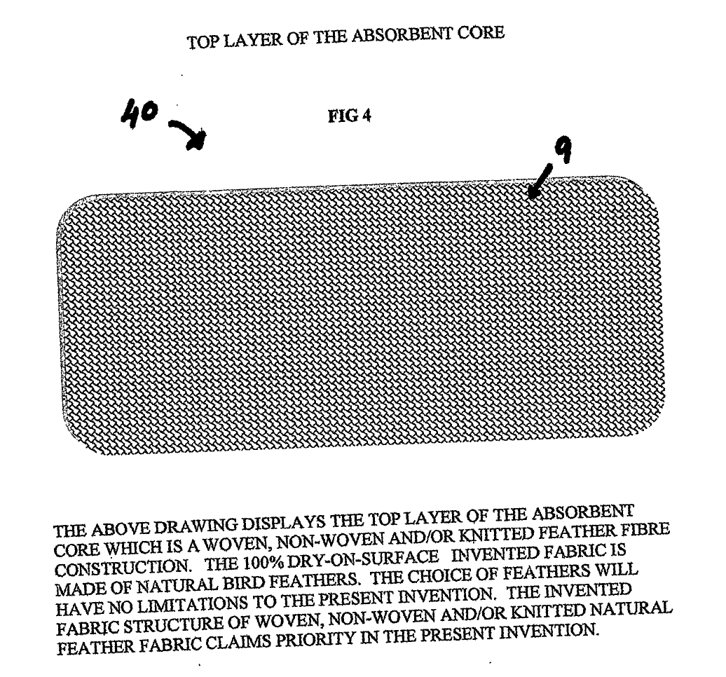

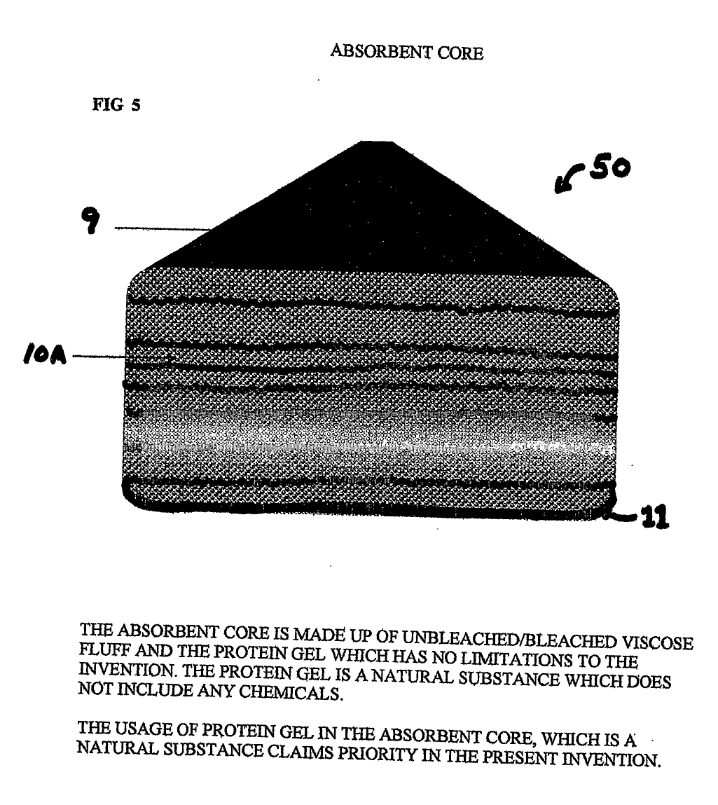

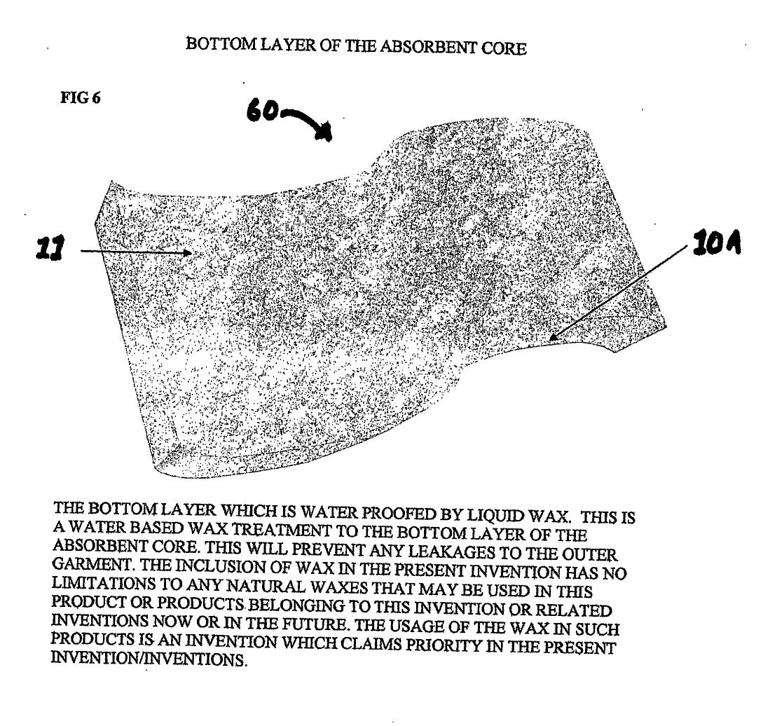

### BACKGROUND OF THE PRESENT INVENTION

The present invention relates to constructions of natural fibres and materials in form of a wiking (top surface layer), an absorbent core and the overall outer and inner materials. These invented structures could be used in baby nappies/diapers, incontinence adult and children's diapers, sanitary wear, medical clothing, bed protectors, chair protectors, baby cot protectors, ribs or any other clothing, used as a complete structure or used as individual fabric constructions, without any limitations in design, structure, method and usage in any application.

Only natural fibres, liquids, waxes, natural protein gels and feathers are used in the present invention. The invented materials may be used individually or used as a complete structure as required. This invention shall not be limited to the products, design, manufacture and manufacturing methods discussed and claimed herein.

Many staple fibres have been used since the history of textile, such as hair alpaca, angora, camel, cashmere, goat, llama, mohair, rabbit, vicuna wool, merino and other types of wool. Staple fibres are in the family of protein fibres and filament fibres. Natural silk may be cultivated or wild. To date there is no evidence of an invented fabric that has been woven, knitted or felted (non-woven) by natural feathers which may be from any bird species. There are no limitations to any species of birds' feathers that could be used in the present invention, now or in the future. History has yet to record an invented fabric structure woven, felted (non-woven) or knitted with feathers. The present invention has a value as a hydrophobic material that will be used in the present invention as a wiking. It could also be used in any other form as a textile material. Feathers are a large global industry used for hats, crafts, and shawls. Once gathered, feathers could be processed in the form of wool, silk, cashmere or in the needle felting method. The very first invented feather yarn or fabric will be produced as woven, nonwoven or knitted as required. The present invention may well provide one of the last missing components to the manufacturing of a 100% bio-degradable product. This would be a global achievement for a safer world. The materials presently used in conventional, disposable nappies have a highly damaging impact on the environment as the plastic/chemical component used therein take hundreds of years to break down.

The invented feather fabric could be used in all forms of clothing. When used as a top layer or wiking in diapers, it would assure 100% dryness next to the skin. This is due to the waxed coating of natural feathers which, as the saying goes, is “like pouring water on a duck's back”. The fabric is used without any limitations on diapers, incontinence garments, bed protectors, chair pads or covers, sanitary products, wipes, fabric for clothing, medical clothing, casual clothing and evening fashion wear. The present invention will however not be limited to the designs, structures and methods of manufacture discussed and claimed herein.

The outer cover in the present invention could be used for baby nappies/diapers, incontinence products and forms of clothing to provide natural air circulation to the skin. The natural fibre construction used in the single use products offers maximum health benefits and ecological sensibility with an ideal balance of comfort. At present, there is global concern about male infertility, nappy rash and other conditions leading to serious illnesses in babies due to the lack of skin breathability. With its unique properties of dryness on the skin, whilst allowing natural air circulation, the present mention could help prevent these and other potentially harmful conditions.

The present invention utilises as its outer cover the construction of a 100% cotton fabric, mated with a 100% cotton felt and has the appearance and touch similar to woven fabric. This ensures a feeling of softness next to the skin. Due to the exclusive use of natural fibres, it is unlikely to cause allergies. It has outstanding absorption properties to wipe-dry water and oil, maintaining good softness, even in wet conditions.

With regard to environmental impact, the present invention reduces destruction of forest resources. It has excellent break-down properties which allow it to be decomposed by micro organisms and returned to nature in a short period of time. It can be easily incinerated without generating any bad odours or toxic gases. The present invention has excellent hygroscopic and moisture retention properties and will not conduct electricity, thereby rendering it safe to use in any form of clothing. The fabric contains a minimum amount of lint and is low pilling. The fabric can undergo various kinds of sterilising treatment, without limitation, the present invention can be used in the manufacture of baby nappies/diapers, incontinence diapers, sanitary napkins, underwear, fashion garments, medical garments, bibs, breast pads, bedding, upholstery and in many other applications. The method of combining the mated fabrics will be in any form compiled by the inventor, now or in the future.

Babies' and infants' skin is a very important factor in considering the design of baby clothing. Outside the mother's womb, a new baby will react to fluctuating temperatures in its new environment. Baby clothing should be soft and pliable and never irritating. It should also be lightweight, yet warm. However it is a must and a very important factor to provide ventilation around the skin to allow moisture to evaporate naturally.

With conventional nappies made from synthetic materials, statistics show that 78% of babies suffer from nappy rash. The main reason for this is lack of air circulation around the skin.

The present invention would be of assistance in dealing with the problem of uncontrollable discharge of urine, faeces or body fluid which is a universal problem in infants as well as patients, both young and old.

The present invention further relates to an absorbent structure and more particularly to a compressed absorbent composite containing natural material which absorbs large quantities of liquid, as detailed below:

Conventional disposable absorbent products have been known to include such products as disposable diapers, sanitary napkins and incontinence pads. All of these products incorporate an absorbent batt which is used to absorb and hold or contain body liquids. The conventional disposable diaper/nappy uses a chemical substance called polyacrylate and more recently the use of corn starch, which is spread in powder form in the pulp to promote absorbancy. Polyacrylate dehydrates the skin and leads to nappy rash and other related illnesses in babies.

The present invention does not include polyacrylate or any form of chemicals. Absorption occurs by the inclusion of a natural absorbent protein gel (in powder form) which is incorporated in the natural fluffed pulp.

The natural fluffed pulp (unbleached or bleached, at the discretion of the inventor) is sprinkled with a protein gel powder which is known as gelatine. Gelatine is a very safe substance which is used in food and prescribed for invalids and babies. Both the protein gel in powder form and the fluffed pulp will be compressed in the method of manufacture.

The present invention also relates to a special method of water proofing. The absorbent core's final layer is treated with a water based wax. The wax is used to waterproof garments, sports gear and umbrellas. The present ion does not include any form of plastics

The inventor requires the product to be 100% bio degradable and baby safe, allowing maximum natural air circulation to the skin thus assuring safety, comfort and warmth which are the three main considerations for baby clothing.

The present mention further relates to using 100% natural soft rubber thread for the gathering of material around the waist and leg. Cotton adhesive tape, and cotton crêpe, satin or sateen, pure silk or cotton decoratives, as well as adhesives, and for fastening together, as best known to the inventor is used. The present invention is not limited as to the natural materials or natural fibres that is used.

One of the present invention's primary purposes is to safeguard the environment. The rapid filling of landfill sites with non-biodegradable plastics used in the conventional disposable nappy is a huge threat globally. Most mothers are concerned about global warming and the environment, yet our present lifestyle demands a product that assures convenience as well as safety for babies and the planet. The present invention meets all the above objectives and is 100% biodegradable.

### THE OBJECTIVES OF THE PRESENT INVENTION

With the present invention the inventor intends to reduce issues such as global warming which could result in ocean rising, land and coast line flooding causing millions of people to abandon their homes, low lying islands disappearing, droughts causing forests to die and food crops to fail, wild life population being wiped out and people facing severe food shortages.

Global warming impacts everywhere, it affects plants, animals and people everywhere. The present invention offers one of the best solutions with potential to minimise ongoing threats human being faced from the effects of global warming. Heat waves, drought and other freak catastrophes of nature are emerging signs.

Designers and inventors play a major role in safeguarding the planet. Warning signs have been ignored by many and the threat is now severe. The inventor does not want to refer to any particular product or products which may have contributed to the present situation on a global scale. However, the inventor wants to assure the world that her present line of products, fabrics, and absorbers will definitely strike a balance between comfort for the user and ecological sensibility for the whole world.

The present invention will allow mothers to purchase a product or products without any guilt, but with a clear conscience that they are supporting efforts to save the planet.

### DISCLOSURE OF THE INVENTION

The present invention disclosed herein is a disposable material of na fibre construction used as an individual fabric or in a combination of structures that is used in diapers, nappies, incontinence products, bibs, bed protectors, medical products, and sanitary products and in any clothing or fashion articles or any other uses and applications now or in the future.

The diaper in particular has an outer cover made from material such as bandage cloth, gauze, leno, sateen or pure silk combined with 100% felt cotton fabric. These materials aim sandwiched together to form the outer cover. The method of sandwiching may be by state of the art machinery or by a process of manual manufacturing.

In the present invention, the top surface layer of the absorbent core is the invented natural feather fabric which can be woven, non-woven or knitted. The natural feather fabric in the present invention is used for the first time in the textile industry. This top layer of natural feather fabric would allow liquids such as body fluids to pass through while still keeping the skin 100% dry at all times.

In the present invention, the inner section of the absorbent core is natural fluffed pulp with the inclusion of a protein gel in powder form. The natural fluffed pulp may be bleached or unbleached as best known to the inventor. The inclusion of the natural fluffed pulp would vary from 0% to 100% and the protein based gel may vary from 0% to 100%. The natural fluffed pulp and the protein gel will be in a compressed form as the main absorber of liquid.

The present invention has a unique waterproofing method used for the impervious outer layer of the structure. The present mention uses a water based wax without limitations to the method or the wax substance used. This waterproofing method may be used in the outer cover or in the final layer of fabric that forms the absorbent core.

The present invention further relates to all natural rubber threads used for elasticity, natural fibre trimmings and adhesive tapes.

The present invention uses one of the natural fabrics for a 100% bio-degradable bag to be used for the disposal of each individual used diaper/nappy.

In the past “super absorbent” materials which absorb several times their weight in liquid have been developed.

Attempts have been made to incorporate these materials in absorbent products such as diapers and sanitary napkins, to enhance their absorbent properties. Perhaps one of the first products to incorporate such absorbent materials is a disposable diaper disclosed in U.S. Pat. No. 3,670,731. This patent discloses an absorbent dressing comprising an absorbent layer sandwiched between a permeable facing and impermeable backing sheet. The absorbent layer contains water insoluble, cross linked, hydrocolloid polymer as the absorbent material.

Although super absorbent materials have been available for many years, they have not gained wide acceptance due to the health issues. The main reason for tis lack of acceptance of superabsorbents is that they are chemically based and could lead to health problems or complications.

The present invention is introduced to the global market to overcome health and environmental problems.

### BRIEF DESCRIPTION OF THE DRAWINGS

Drawing 1 (10) is a perspective view illustrating two layers of materials in the present invention. No 1 is made of bandage cloth or gauze, or leno or sateen cloth or pure silk. No 2 represents the cotton felt construction. These two layers form the outer cover of the diaper/nappy or its related products.

Drawing 2 (20) a perspective view of the completed diaper/nappy. No 3 represents the cotton adhesive tape. No 4 represents the cotton crêpe band. No 5 represents the outer cover. No 6 represents the soft edging.

Drawing 3 (30) a perspective view of the reverse side of the diaper/nappy. No 5 represents the outer cover. No 8 represents the brand label. (No 8 may or may not be included as best known to the inventor.

Drawing 4 (40) a perspective view of the absorbent core which is placed at the centre of the diaper/nappy. No 9 represents the top layer of the invented natural feather construction of woven, non-woven or knitted fabric.

Drawing 5 (50) a 3D view of the invented absorbent core. No 9 represents the top layer of the natural feather invented fabric. No 10A represents the natural fluffed pulp with the inclusion of the protein gel. The natural fluffed pulp and the protein gel is compressed in the method of manufacturing.

Drawing 6 (60) a 3d view of the invented absorbent core displaying the bottom layer of the absorbent core which is represented by No 11. No 11 is the wax treated layer for water proofing of the nappy or diaper. 10A is the absorbent natural fluffed pulp and the natural gel.

1.-45. (canceled)

46. A multi-layered composition comprising:
a sandwiched outer cover of natural fabric and cotton felt, and
an absorbent core comprising a top layer of a natural feather fabric and a lower layer of a waterproof coating of wax.

47. A composition according to claim 46 wherein the feather fabric has a form selected from a group consisting of woven, non-woven, felted and knitted fabric.

48. A composition according to claim 46 wherein the feather fabric is compressed to form a compressed structure.

49. A composition according to claim 46 wherein the feather fabric is formed of bird feathers.

50. A composition according to claim 46 wherein the feather fabric has a weight of 0.02-350 grams per square meter.

51. A composition according to claim 46 wherein the natural fabric comprises at least one of a group consisting of bandage cloth, gauze, cotton, fibre, leno, sateen and pure silk.

52. A composition according to claim 51 wherein the cotton is a 100% cotton fabric.

53. A composition according to claim 46 wherein the cotton felt is a lightweight, non-woven cotton felt.

54. A composition according to claim 46 wherein the natural fabric comprises a 100% cotton fabric mated with a 100% cotton felt.

55. A composition according to claim 46 wherein the absorbent core further comprises a second layer.

56. A composition according to claim 55 wherein the second layer comprises natural fluffed pulp.

57. A composition according to claim 56 wherein the fluffed pulp is bleached or unbleached.

58. A composition according to claim 56 wherein the fluffed pulp is viscose.

59. A composition according to claim 55 wherein the second layer comprises natural protein gel.

60. A composition according to claim 59 wherein the protein gel is gelatine.

61. A composition according to claim 59 wherein the protein gel is in powdered form.

62. A composition according to claim 55 wherein the second layer comprises a compressed mixture of the natural fluffed pulp and a natural protein gel.

63. A composition according to claim 62 wherein the protein gel is in powdered form.

64. A composition according to claim 63 wherein the powdered protein gel is incorporated in the fluffed pulp.

65. A composition according to claim 46 wherein the wax is a natural wax.

66. A composition according to claim 46 wherein the wax is a water based wax.

67. A composition according to claim 46 wherein the wax is a liquid wax.

68. A composition according to claim 46 wherein the wax is beeswax.

69. A composition according to claim 46 wherein the outer cover has a weight of 0.02 to 300 grams per square meter.

70. A composition according to claim 46 wherein the outer cover is formed of breathable material.

71. A composition according to claim 46 wherein a waterproofing treatment is provided in at least one of the outer cover and the absorbent core.

72. A composition according to claim 46 wherein the composition is disposed in a structure, the structure adapted to at least partially absorb waste body fluids of a user.

73. A composition according to claim 72 wherein the structure is in the form of a diaper comprising two leg apertures through which the legs of the wearer are positioned and an abdomen aperture through which the abdomen of the wearer is positioned, the structure having gathering around the leg aperture and the abdomen aperture, the gathering formed of 100% natural soft rubber thread.

74. A composition according to claim 73 wherein the structure further comprises adhesive fastening tapes to secure the diaper in position on the wearer, the fastening tapes formed of natural rubber and fibers.

75. A composition according to claim 72 wherein the composition is in the form of a panel in the structure.

76. A composition according to claim 72 wherein the structure is any one of a group consisting of a diaper, a nappy, a sanitary pad, an incontinence pad or diaper, a sanitary wear, medical clothing, a bed protector, a chair protector, a baby cot protector and a bib.

77. A personal product, clothing item or protective item for at least partially absorbing waste body fluids comprising:
a multi-layered composition, the multi-layered composition including, a sandwiched outer cover of natural fabric and cotton felt, and an absorbent core including a top layer of natural feather fabric and a lower layer of a waterproof coating of wax.

78. A personal product, clothing item or protective item according to claim 77 wherein the feather fabric has a form selected from a group consisting of woven, non-woven, felted and knitted fabric.

79. A personal product, clothing item or protective item according to claim 77 wherein the feather fabric is compressed to form a compressed structure.

80. A personal product, clothing item or protective item according to claim 77 wherein the absorbent core further comprises a second layer.

81. A personal product, clothing item or protective item according to claim 80 wherein the second layer comprises natural fluffed pulp.

82. A personal product, clothing item or protective item according to claim 80 wherein the second layer comprises natural protein gel.

83. A personal product, clothing item or protective item according to claim 80 wherein the second layer comprises a compressed mixture of natural fluffed pulp and natural protein gel.

84. A method of preparing a multi-layered composition for a personal product, clothing item or protective item to at least partially absorb waste body fluids comprising:
i) forming an outer cover by sandwiching together natural fabric and cotton felt;
ii) forming an absorbent core including a top layer of natural feather fabric and a lower layer of a waterproof coating of wax; and
iii) compressing the components resulting from steps i) and ii) to form the composition.

85. A method according to claim 84 wherein the feather fabric is selected from a group consisting of woven, non-woven, felted and knitted fabric.

86. A method according to claim 84 wherein the feather fabric is compressed to form a compressed structure.

87. A method according to claim 84 wherein the absorbent core further comprises a second layer.

88. A method according to claim 42 wherein the second layer comprises natural fluffed pulp.

89. A method according to claim 42 wherein the second layer comprises natural protein gel.

90. A method according to claim 42 wherein the second layer comprises a compressed mixture of natural fluffed pulp and natural protein gel.
